(19) United States
(12) Patent Application Publication
(10) Pub. No.: US 2022/0062163 A1
(43) Pub. Date: Mar. 3, 2022

(54) EFFERVESCENT TABLETS

(71) Applicant: PHARMACANNIS LABS LLC, Oak Park, IL (US)

(72) Inventors: David Schaible, Ulster Park, NY (US); Christopher Diorio, Campbell Hall, NY (US)

(73) Assignee: PHARMACANNIS LABS LLC, Oak Park, IL (US)

(21) Appl. No.: 17/463,842

(22) Filed: Sep. 1, 2021

Related U.S. Application Data
(60) Provisional application No. 63/072,981, filed on Sep. 1, 2020.

(51) Int. Cl. A61K 9/46; A61K 47/26; A61K 47/14; A61K 31/05; A61K 31/352; A61K 47/12

(52) U.S. Cl. CPC A61K 9/0007; A61K 47/26; A61K 47/12; A61K 31/05; A61K 31/352; A61K 47/14

(57) ABSTRACT

Effervescent self-emulsifying drug delivery system formulations for oral administration of water-insoluble cannabinoids are disclosed.

### FIELD OF THE INVENTION

The present invention relates to effervescent tablet formulations for the delivery of cannabinoids.

### BACKGROUND OF THE INVENTION

Public interest in the medicinal use of cannabis has grown exponentially over the past decade. Cannabis sativa is an annual plant belonging to the Cannabaceae family. It contains more than 400 chemicals and approximately 80 cannabinoids, the active constituents of cannabis, including tetrahydrocannabinol (THC), cannabidiol (CBD), cannabinol (CBN), tetrahydrocannabivarin (THCV) and cannabigerol (CBG). Pharmacologically, the principal psychoactive constituent of cannabis is tetrahydrocannabinol (THC), which is used for treating a wide range of medical conditions, including glaucoma, AIDS wasting, neuropathic pain, treatment of spasticity associated with multiple sclerosis, fibromyalgia and chemotherapy-induced nausea. THC is also effective in the treatment of allergies, inflammation, infection, epilepsy, depression, migraine, bipolar disorders, anxiety disorder, drug dependency and drug withdrawal syndromes.

Additional pharmacologically-active cannabinoids include cannabidiol (CBD), an isomer of THC, which is a potent antioxidant and anti-inflammatory compound known to provide protection against acute and chronic neurodegeneration; cannabigerol (CBG), found in high concentrations in hemp, which acts as a high affinity a2-adrenergic receptor agonist, moderate affinity 5-HT_{1A} receptor antagonist and low affinity CB1 receptor antagonist, and possibly has anti-depressant activity; and cannabichromene (CBC), which possesses anti-inflammatory, anti-fungal and anti-viral properties. Many cannabinoids have therapeutic potential in a variety of diseases and may play a relevant role in pharmacology.

The primary method used to deliver marijuana into a patient's system is by smoking the marijuana. However, smoking increases an individual's risk for cancer, lung damage and emphysema. Furthermore, since marijuana does contain high levels of the psychoactive drug Δ⁹-THC, there has been considerable debate whether the potential health benefits of smoking marijuana are outweighed by the associated health risks.

Oral administration is the easiest and most convenient route for non-invasive drug administration. However, cannabinoids are highly lipophilic, meaning that they are soluble in lipids and some organic solvents while being substantially insoluble or only sparsely soluble in water. Cannabinoids are soluble in highly non-polar solvents (i.e., in substances such as chloroform, dichloromethane and high concentrations of alcohol); they also have limited solubility in glycols. Some of these solvents are pharmaceutically unacceptable, and the pharmaceutically acceptable solvents need to be used in high concentrations to produce solutions. Moreover, solubility in some of these solvents imposes a ceiling on the dose that can be given using conventional pharmaceutical methods of formulation. As such, the poor water-solubility of cannabinoids results in major difficulties in formulation and presents a major challenge to consistent drug delivery.

Furthermore, when administered orally in the form of an oil solution or some kind of water and/or oil suspension or emulsion, lipophilic compounds usually show poor bioavailability. For example, Δ⁹-THC is almost completely absorbed (90% to 95%) after a single oral dose. However, due to the combined effect of first pass, hepatic metabolism, and high lipid solubility, only about 10% to 20% of an administered dose reaches systemic circulation.

Another impediment to the medicinal use of cannabis is its well-known psychotropic side effects. Recent developments suggest that several cannabinoids exert very weak or no psychotropic effects. These include cannabidiol (CBD), cannabigerol (CBG), cannabichromene (CBC), Δ⁹-tetrahydrocannabivarin (Δ⁹-THCV), cannabidivarin (CBDV) as well as cannabinoid acids such as Δ⁹-tetrahydrocannabinolic acid (Δ⁹-THCA) and cannabidiolic acid (CBDA). These non-psychotropic cannabinoids have been shown to exert a wide range of pharmacological effects and could potentially be used to produce formulations with reduced or no psychotropic side effects.

Accordingly, there is a need for developing oral formulations of cannabinoids with enhanced bioavailability. Given the numerous cannabinoids with pharmacological activities, there is also a need to develop formulations with different ratios of two or more cannabinoids. Additionally, there is a need for developing cannabinoid formulations with reduced or no psychotropic side effects.

### SUMMARY OF THE INVENTION

The invention described in the following embodiments provides a cannabinoid self-emulsifying drug delivery system (SEDDS), preferably in the form of an effervescent tablet, providing enhanced bioavailability compared to the same cannabinoid administered orally in the form of an oil solution or a water/oil suspension or emulsion, while decreasing intra- and inter-patient variability in observed blood levels of cannabinoids.

The present inventors discovered, inter alia, that cannabinoid-containing oil and a single surfactant, preferably Vitamin E TPGS, are sufficient to form a self-emulsifying drug delivery system (SEDDS) that can be adsorbed onto a suitable powder (e.g. an acid and/or carbon dioxide source and/or a compressible sugar) and formulated into effervescent formulations with superior bioavailability. The present inventors further discovered that compressible sugars (e.g. compressible sucrose) have a suitable surface area and particle size to adsorb oily cannabinoid compounds onto their surface while retaining compaction properties. The present inventors further discovered that coating of the SEDDS particles with colloidal silicon dioxide improves manufacturability and compression properties.

In some embodiments, a free-flowing, compressible effervescent self-emulsifying drug delivery system (SEDDS) powder is provided, the effervescent SEDDS powder comprising a cannabinoid-containing oil (e.g. cannabinoid extract), a suitable surfactant, an acid source in powder form, and a carbon dioxide source in powder form. In preferred embodiments, the surfactant is Vitamin E TPGS. In some aspects, the powder is produced by mixing liquid cannabinoid-containing oil and Vitamin E TPGS with an acid source and a carbon dioxide source (the acid and carbon dioxide sources in powder form) in a mixer (e.g. a centrifugal planetary mixer) for a suitable period of time until the oil is completely adsorbed to the powder.

The cannabinoid oil may be prepared according to methods known in the art. In preferred embodiments, the cannabinoid oil is prepared by supercritical CO₂ or polar solvent (preferably ethanol) extraction of the desired cannabinoids from plant material. In some embodiments, the cannabinoid oil comprises between about 40% and 95% w/w cannabinoids.

Suitable sources of carbon dioxide include, without limitation, sodium bicarbonate, sodium carbonate, potassium carbonate, potassium carbonate and calcium carbonate. Preferably, the carbon dioxide source is in powder form.

Suitable acid sources can be obtained from acids, acid anhydrides and acid salts and include, without limitation, citric acid, tartaric acid and ascorbic acid. Preferably, the acid source is in powder form.

Suitable surfactant/lipophilic carriers include water soluble nonionic surfactant that exhibit amphipathic properties, preferably with emulsifying, dispersing, gelling and/or solubilizing effects on poorly water-soluble compounds. In preferred embodiments, the surfactant/lipophilic carrier is vitamin E TPGS (D-α-Tocopheryl polyethylene glycol succinate) and the SEDDS powder does not comprise any other surfactants. Vitamin E TPGS has nonionic surfactant-like properties and exhibits amphipathic properties with emulsifying, dispersing, gelling and solubilizing effects on poorly water-soluble compounds. Vitamin E TPGS can also act as a P-glycoprotein (P-gp) inhibitor, which can help to improve bioavailability and can also serve as an antioxidant in vivo.

In preferred embodiments, the SEDDS effervescent powder (comprising a cannabinoid-containing oil and a suitable surfactant adsorbed onto an acid source in powder form, and a carbon dioxide source in powder form) is mixed (coated) with colloidal silicon dioxide. Thus, in some embodiments, an SEDDS effervescent powder is provided comprising a cannabinoid-containing oil, a surfactant, an acid source, a carbon dioxide source and colloidal silicon dioxide.

In other preferred embodiments, the SEDDS powder (comprising a cannabinoid-containing oil and a surfactant adsorbed onto an acid source and a carbon dioxide source and colloidal silicon dioxide) is mixed with a compressible sugar, nonlimiting examples of which include compressible sucrose, compressible lactose and compressible dextrose. Preferably, the compressible sugar is compressible sucrose. Compressible sucrose contains between 95 and 98% of sucrose and an additive such as starch, maltodextrine, inert sugar or a lubricant, e.g. Di-Pac® from Domino Sugar Corp.

In some embodiments, the SEDDS powder further comprises a lubricant. In a preferred embodiment, the SEDDS powder comprises the lubricant sodium stearyl fumarate.

In some embodiments, the SEDDS powder further comprises a flavoring agent. In preferred embodiments, the SEDDS powder comprises lemon and/or orange oil as flavoring agent(s).

In related embodiments, an SEDDS effervescent powder as herein described (comprising a cannabinoid-containing oil and a surfactant adsorbed onto an acid source and a carbon dioxide source and colloidal silicon dioxide and optionally further comprising a compressible sugar and/or lubricant and/or flavoring agent) is formulated into an effervescent tablet. In preferred embodiments, an SEDDS effervescent tablet is provided comprising comprising (i) a cannabinoid-containing oil and Vitamin E TPGS adsorbed onto an acid source and a carbon dioxide source (ii) colloidal silicon dioxide (iii) compressible sucrose (iv) sodium stearyl fumarate and (v) lemon and/or orange oil. The effervescent tablet self emulsifies in a liquid such as water prior to patient consumption and is immediately available for absorption upon entering the gastrointestinal tract without the need for tablet disintegration and emulsification in the body.

In some embodiments, the cannabinoid is present in the SEDDS effervescent tablet at a dose of from 1 to 100 mg, more preferably from 2.5 to 75 mg, more preferably from 2.5 to 50 mg, more preferably from 2.5 to 25 mg, more preferably from 2.5 to 20 mgs, from about 1.7 mg to about 9.5 mg or about 10 mg, 9.5 mg, 9 mg, 8.5 mg, 8 mg, 7.5 mg, 7 mg, 6.5 mg, 6 mg, 5.5 mg, 5 mg, 4.5 mg, 4 mg, 3.5 mg, 3 mg, 2.5 mg, 2 mg, 1.5 mg or 1 mg.

In some embodiments, the SEDDS effervescent tablet comprises vitamin E TPGS at a weight/weight percentage of between 0.25% and 0.5%, between 0.3% and 0.45%, between 0.35% and 0.4% and preferably about 0.37%.

In some embodiments, the SEDDS effervescent tablet comprises compressible sucrose at a weight/weight percentage of between 15% and 25%, more preferably between 17% and 23%, more preferably at about 21.20%.

In some embodiments, the SEDDS effervescent tablet comprises colloidal silicon dioxide at a weight/weight percentage of between 0.18% and 0.30%, preferably between 0.20% and 0.28%, more preferably between 0.22% and 0.26%, more preferably at about 0.24%.

In some embodiments, the SEDDS effervescent tablet comprises citric acid (as an acid source) at a weight/weight percentage of between 37% and 47%, between 39% and 45%, between 41% and 43% or about 42%.

In some embodiments, the SEDDS effervescent tablet comprises sodium bicarbonate as a carbon dioxide source, present at a weight/weight percentage of between 28% and 40%, between 30% and 38%, between 32% and 36% or about 34%.

In some embodiments, the SEDDS effervescent tablet comprises a flavoring agent at a weight/weight percentage of about 0.1% to about 0.6%, about 0.2% to about 0.5%, 0.3% to about 0.4% or about 0.37%.

In some embodiments. The SEDDS effervescent tablet comprises sodium stearyl fumarate, which may be present at a weight/weight percentage of between 0.75% and 2.25%, between 1.0% and 2.0% or about 1.5%.

In particularly preferred embodiment, the SEDDS effervescent tablet has the properties of Table 1:

| | | | Colloidal | Sodium | Lemon/ | | | Sodium |
|---|---|---|---|---|---|---|---|---|
| | | Compressible | Silicon | Bicarbonate/ | Orange | Citric | Vitamin E | Stearyl |
| THC | CBD | sucrose | Dioxide | carbonate | Oil | Acid | TPGS | Fumarate |
| mg/dose | mg/dose | % wt/wt | % wt/wt | % wt/wt | % wt/wt | % wt/wt | % wt/wt | % wt/wt |
| <0.5 | 5.0 | 21.20% | 0.24% | 42.00% | 0.37% | 34.00% | 0.37% | 1.50% |
| 5.0 | <0.5 | 21.20% | 0.24% | 42.00% | 0.37% | 34.00% | 0.37% | 1.50% |

An SEDDS effervescent powder as herein described may be compressed into an effervescent tablet using direct compression or any suitable method as will be appreciated by the skilled person.

The cannabinoid SEDDS effervescent formulations as described herein may be useful in the treatment and/or prevention of a disease, condition, or symptom of a disease including, but not limited to, glaucoma, AIDS wasting, neuropathic pain, treatment of spasticity associated with multiple sclerosis, post-traumatic stress disorder, fibromyalgia and chemotherapy-induced nausea, allergies, inflammation, infection, epilepsy, depression, migraine, bipolar disorders, anxiety disorder, drug dependency and drug withdrawal syndromes.

SEDDS effervescent tablets as herein will also help improve patient compliance, being preferable in subject with difficulty in swallowing.

Within the meaning of the present invention is meant by the term “self-emulsifiable mixture”, also known as pre-emulsion or microemulsion pre-mixture comprises a lipophilic phase with at least one surfactant, said mixture capable of forming an emulsion by simple mechanical stirring with an aqueous phase. A self-emulsifiable mixture according to the invention may, upon oral administration, form emulsions with the hydrophilic phase of the organism.

### DETAILED DESCRIPTION OF THE INVENTION

Provided herein is a cannabinoid SEDDS effervescent formulation, in the form of a free flowing powder or tablet that overcomes the low bioavailability of conventional oral dosage forms that employ cannabis extract or distillate and decreases intra- and inter-patient variability in observed blood levels of cannabinoids.

The food effect on the pharmacokinetic profile of typical of oral dosage forms that employ cannabis is minimized by the effervescent tablets described herein. Thus, an effervescent tablet as described herein may exhibit an improved pharmacokinetic profile when consumed with food compared to conventional oral dosage forms containing the same dosage of active ingredient with respect one or more of the following: (i) delay in median Tₘₐₓ (ii) decrease in Cmax and (iii) increase in total exposure (AUC_{inf}).

Compression of effervescent tablets is often complicated due to hygroscopicity of the raw materials (e.g. citric acid and sodium bicarbonate). Hygroscopic compounds typically lead to picking and sticking problems during compression as well as premature effervescence within the tablet matrix after compression, leading to physical and chemical stability problems. This effect is complicated further if cannabis extracts are included in the matrix. Extreme compression difficulties will be encountered due to the oily nature of the extracts and will negatively affect excipient binding and ultimately tablet hardness as well as exacerbating picking, sticking and capping during tablet compression. Effervescent tablets as herein described overcome these issues by loading cannabis extract and surfactant onto a powder carbon dioxide source and/or a powder acid source and/or compressible sucrose and subsequently coating mixture with colloidal silicon dioxide. Thus, effervescent tablets as herein described can be manufactured using conventional direct compression tableting methods

Self-Emulsifying Drug Delivery System

Self-Emulsifying Drug Delivery System (SEDDS) is a solid or liquid dosage form comprising an oil phase, a surfactant and a co-surfactant, characterized primarily in that said dosage form can form oil-in-water emulsion spontaneously in the gastrointestinal tract or at ambient temperature (referring generally to body temperature, namely 37° C.) with mild stirring. When a SEDDS enters the gastrointestinal tract, it is initially self-emulsified as emulsion droplets and rapidly dispersed throughout the gastrointestinal tract, and thus reducing the irritation caused by the direct contact of the drug with the mucous membrane of the gastrointestinal tract. In the gastrointestinal tract, the structure of the emulsion micro particulates will be changed or destroyed. The resulting micro particulates of micrometer or nanometer level can penetrate into the mucous membrane of the gastrointestinal tract, and the digested oil droplets enter the blood circulation, thereby significantly improving the bioavailability of the drug. The self-emulsifying drug delivery system is predominantly employed with respect to lipid-soluble and less water-soluble drugs, such as cannabinoids. It increases the stability and the bioavailability of the lipophilic drugs, provides a more consistent temporal profile of drug absorption, protects the drugs from the hostile environment in the gastro-intestinal tract, eliminates food effects, and allows for dose escalation, thereby improving efficacy and safety.

Cannabinoids

Cannabinoids are a group of extracellular signaling molecules. Signals from these molecules are mediated in animals by two G-protein coupled receptors, Cannabinoid Receptor 1 (CB₁) and Cannabinoid Receptor 2 (CB₂). CB₁ is expressed most abundantly in the neurons of the central nervous system (CNS) but is also present at lower concentrations in a variety of peripheral tissues and cells (Matsuda, et al. (1990) Nature 346:561-564). In contrast, CB₂ is expressed predominantly, although not exclusively, in non-neural tissues, e.g., in hematopoietic cells, endothelial cells, osteoblasts, osteoclasts, the endocrine pancreas, and cancerous cell lines (Munro, et al. (1993) Nature 365:61-65; and as reviewed in Pacher, et al. (2006) Pharmacol. Rev. 58(3): 389-462). As such, CB₁ is believed to be primarily responsible for mediating the psychotropic effects of cannabinoids on the body, whereas CB₂ is believed to be primarily responsible for most of their non-neural effects.

The well-known psychotropic effects of Δ⁹-THC have greatly limited its clinical use. However, as described above, the plant Cannabis contains many cannabinoids with weak or no psychoactivity that, therapeutically, might be more promising than Δ⁹-THC, or can be combined with lower doses of Δ⁹-THC to produce equivalent therapeutic benefits. The cannabinoid SEDDS of the present invention are helpful in addressing the adverse psychotropic effects of Δ⁹-THC.

In some embodiments, the cannabinoid is selected from tetrahydrocannabinol, Δ⁹-tetrahydrocannabinol (THC), Δ⁸-tetrahydrocannabinol, Δ⁸-tetrahydrocannabinol-DMH, Δ⁹-tetrahydrocannabinol propyl analogue (THCV), 11-hydroxy-tetrahydrocannabinol, 11-nor-9-carboxy-tetrahydrocannabinol, 5′-azido-Δ⁸-tetrahydrocannabinol, AMG-1, AMG-3, AM411, AM708, AM836, AM855, AM919, AM926, AM938, cannabidiol (CBD), cannabidiol propyl analogue (CBDV), cannabinol (CBN), cannabichromene, cannabichromene propyl analogue, cannabigerol, CP 47497, CP 55940, CP 55244, CP 50556, CT-3 (ajulemic acid), dimethylheptyl HHC, HU-210, HU-211, HU-308, WIN 55212-2, desacetyl-L-nantradol, dexanabinol, JWH-051, levonantradol, L-759633, nabilone, O-1184, and mixtures thereof.

### EXAMPLES

The following examples illustrate preferred embodiments of the present invention and are not intended to limit the scope of the invention in any way. While this invention has been described in relation to its preferred embodiments, various modifications thereof will be apparent to one skilled in the art from reading this application.

A list of materials used in the Examples and the source of these materials is as provided in Table 2:

| | Specifi- | | Brand | |
|---|---|---|---|---|
| Ingredient | cation | Function | Name | Supplier |
| Compressible | NF | Binder | DiPac | Domino |
| Sucrose | | | | Antares |
| | | | | Health |
| Vitamin E TPGS | NF | lipophilic carrier | NA | Products |
| Colloidal Silicon | NF | Adsorbent | Aerosil 300 | Evonik |
| Dioxide | | | | |
| Lemon/Orange Oil | NF | flavor | NA | PENTA |
| Citric Acid | NF | Effervescent | NA | Sigma, |
| | | | | EMD |
| Sodium | USP/NF | Effervescent | Effersoda | SPI |
| Bicarbonate/ | | | | Pharma |
| carbonate | | | | |
| Sodium Stearyl | NF | Lubricant | Pruv | JRS |
| Fumarate | | | | Pharma |

### Example 1—General Method for Preparing a Cannabinoid SEDDS Effervescent Tablet

A cannabinoid SEDDS effervescent tablet as described herein may be according to the following steps:

Step 1: Dried cannabis material is soaked in super-cooled ethanol (−70 C) for about 30 minutes.

Step 2: The slurry (cannabis and ethanol) from step 1 is centrifuged to separate ethanol (containing cannabinoids) from plant material and subsequently filtered to remove particulate plant material

Step 3: The ethanol containing the cannabinoids is heated and a vacuum is applied in a falling film evaporator or rotovap to separate the cannabinoids from the ethanol.

Step 4: The resulting oil from step 3 is heated to 120 C for up to 4 hours to decarboxylate the cannabinoids (e.g. THCA to free THC)

Step 5: Material from step 4 is then treated to purify and concentrate the cannabinoids contained in the oil. For example, the material from step 4 may be distilled at about 185 C under vacuum.

Step 6A: The resulting cannabinoid oil is then mixed with Vitamin E TPGS and optionally a flavoring agent (e.g. lemon oil).

Step 6B: The mixture from step 6A is then blended with a suitable powder carbon dioxide source (e.g. sodium bicarbonate powder such as Effer-Soda®) and a suitable powder acid source (e.g. citric acid) in a centrifugal planetary mixer.

Step 7: The resulting powder from step 6B is then mixed in a suitable powder blender with colloidal silicon dioxide.

Step 8: The resulting powder from step 7 is then mixed with compressible sucrose and sodium stearyl fumarate.

The final blend from step 8 is then compressed into tablets on a suitable rotary tablet press

### Example 2—Determination of Oral Bioavailability I

Subjects are selected for the in vivo oral bioavailability study. Subjects are fasted overnight prior to dosing. An SEDDS formulation (e.g. in the form of an effervescent tablet) as herein described is orally administered to a first group of subjects (n=10). The same dose of cannabinoids is administered orally to second group of subjects (n=10) in the form of an oil solution. The same dose of cannabinoids is administered intravenously to third group of subjects (n=10).

Serial blood samples of 2 mL are obtained from subjects at 20 and 40 minutes and 1, 2, 4, 6, 8, 12, and 24 hours after dosing. These blood samples are analyzed using an HPLC or LC/MS/MS assay specific for the cannabinoids administered to each subject.

Samples are typically prepared by adding 25 μL aliquots of plasma to 200 μL of a solution of 0.1% formic acid in acetonitrile:methanol 1:1 containing THC-D₃ and 11-hydroxy THC-D₃ and extracted through a Biotage Isolute PLD+ plate (50 mg) extraction plate (Biotage LLC, Charlotte, N.C.). Extracted samples are analyzed by reverse phase liquid chromatography/tandem mass spectrometry (LC/MS/MS) in selective reaction monitoring (SRM) mode under optimized positive ion conditions for the detection of THC, 11-hydroxy THC and the deuterated internal standards. Analytes are separated by reverse phase HPLC employing a Waters BEH C8 (2.1×30 mm) column (Waters Corp., Milford, Mass.), under gradient conditions. The gradient begins with 50% MPA (10 mM ammonium acetate in water pH 4.8) for 0.5 minutes then increases to 95% MPB (0.1% acetic acid in methanol) in linear fashion over 1.5 minutes and then holds at 95% MPB for 1 minute before returning to the starting conditions. For MS/MS detection a Sciex API 5000 mass spectrometer (AB Sciex LLC, Redwood City, Calif.) is used to monitor four transition ions (THC 315.1→193.0, THC-D₃ 318.1→196.0, 11-hydroxy 331.1→193.0 and 11-hydroxy D₃ 334.1→196.0). The targeted quantitation range is 0.1 ng/mL to 100 ng/mL for THC and from 0.2 ng/mL to 200 ng/mL for 11-hydroxy THC.

Drug concentrations in the blood of the test subjects are plotted against the time after the drug is administered through an intravenous (iv) or oral route. The area under the plasma concentration-time curve (the AUCs) are recorded and integrated using the trapezoidal rule to calculate the absolute bioavailability according to the following formulae:

$$Absolute bioavailability ( \% ) = \frac{( AUC ) oral \text{/} Dose oral}{( AUC ) iv \text{/} Dose iv}$$

The self-emulsifying drug delivery system as herein described achieves an oral bioavailability of the cannabinoids significantly higher than the same dose of cannabinoids administered orally in the form of an oil solution.

### Example 3—Determination of Oral Bioavailability II

The plasma pharmacokinetics of a cannabinoid SEDDS effervescent tablet formulation as herein described and a commercially available THC tablet are measured in a study utilizing non-naïve male Beagle dogs (n=4 for each test compound). In these tests the SEDDS formulation comprises 5 mg/dose THC, 21.20% wt/wt compressible sucrose, 0.24% wt/wt colloidal silicon dioxide, 42% wt/wt sodium bicarbonate/carbonate, 0.37% wt/wt lemon/orange oil, 34% wt/wt citric acid, 0.37% wt/wt vitamin E TPGS and 1.50% wt/wt sodium stearyl fumarate. The commercially available tablet formulation also contains 5 mg THC as an active ingredient. A 3 ml blood sample is drawn from each subject dog prior to oral administration of a single dose of the test compound. Blood samples are also taken at 0.5, 1, 2, 4, 6, 8, and 12 hours post-administration. Plasma is isolated from each sample and each sample is split into two equal aliquots and stored at −80° C. until further analysis.

1. A self-emulsifying drug delivery system (SEDDS) in the form of a free-flowing, compressible effervescent powder, comprising (i) a cannabinoid-containing oil and a surfactant adsorbed onto an acid source and a carbon dioxide source, wherein the acid source and carbon dioxide source are in powder form.

2. The SEDDS powder of claim 1, wherein the surfactant is Vitamin E TPGS and wherein the SEDDS powder does not comprise any other surfactants.

3. The SEDDS powder of claim 1, wherein the acid source is selected from the group consisting of citric acid, tartaric acid and ascorbic acid citric acid; and wherein the carbon dioxide source is selected from the group consisting of sodium bicarbonate, sodium carbonate, potassium carbonate, potassium carbonate and calcium carbonate.

4. The SEDDS powder of claim 1, further comprising (ii) colloidal silicon dioxide

5. The SEDDS powder according to claim 1, further comprising (iv) a compressible sugar.

6. The SEDDS powder according to claim 5, wherein the compressible sugar is compressible sucrose.

7. The SEDDS powder according to claim 1, further comprising one or more of (v) a flavoring agent and (vi) lubricant.

8. The SEDDS powder according to claim 7, wherein the flavoring agent is lemon or orange oil and wherein the lubricant is sodium stearyl fumarate.

9. The SEDDS powder according to claim 1, wherein the cannabinoid-containing oil comprises at least one cannabinoid selected from the group consisting of tetrahydrocannabinol (THC), cannabidiol (CBD), cannabigerol (CBG), cannabichromene (CBC), Δ9-tetrahydrocannabivarin (Δ9-THCV), cannabidivarin (CBDV), Δ9-tetrahydrocannabinolic acid (Δ9-THCA), and cannabidiolic acid (CBDA).

10. The SEDDS according to claim 9, wherein the at least one cannabinoid is one or more of THC and CBD.

11. An effervescent tablet comprising the SEDDS powder of claim 1.

12. The effervescent tablet according to claim 11, wherein the acid source comprises citric acid.

13. The effervescent tablet according to claim 11, wherein the carbon dioxide source comprises sodium bicarbonate.

14. The effervescent tablet of claim 11, wherein THC is present in the tablet in an amount between about 1 mg and about 100 mg, between about 1 mg and about 50 mg, between about 1 mg and about 25 mg, between about 1 mg and about 15 mg, between about 1 mg and about 10 mg, or between about 1 mg and about 5 mg; or is present at about 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 20 mg, 20.5 mg, 21 mg, 21.5 mg, 22 mg, 22.5 mg, 23 mg, 23.5 mg, 24 mg, 24.5 mg, or 25 mg.

15. The effervescent tablet according to claim 11, wherein CBD is present in the tablet at a concentration of between about 1 mg and about 100 mg, between about 1 mg and about 50 mg, between about 1 mg and about 25 mg, between about 1 mg and about 15 mg, between about 1 mg and about 10 mg, or between about 1 mg and about 5 mg; or is present at about 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 20 mg, 20.5 mg, 21 mg, 21.5 mg, 22 mg, 22.5 mg, 23 mg, 23.5 mg, 24 mg, 24.5 mg, or 25 mg.

16. The effervescent tablet according to claim 11, comprising about 15% wt/wt to about 25% wt/wt compressible sucrose, about 0.15% wt/wt to about 0.35% wt/wt colloidal silicon dioxide, about 30% wt/wt to about 50% wt/wt sodium bicarbonate/carbonate, about 25% wt/wt to about 35% wt/wt citric acid, about 0.15% wt/wt to about 0.5% wt/wt Vitamin E TPGS and from about 0.75% to about 2.25% wt/wt sodium stearyl fumarate.

17. The effervescent tablet according to claim 16, comprising about 21.20% wt/wt compressible sucrose, about 0.24% wt/wt colloidal silicon dioxide; about 42% wt/wt sodium bicarbonate/carbonate, about 34% wt/wt citric acid, about 0.37% vitamin E TPGS and about 1.5% sodium stearyl fumarate.

18. The effervescent tablet according to claim 16 comprising about 0.1% to about 0.5% flavoring agent.

19. The effervescent tablet according to claim 16 comprising one or more of about 5 mg THC and about 5 mg CBD.
